(19) United States
(12) Patent Application Publication
(10) Pub. No.: US 2023/0062528 A1
(43) Pub. Date: Mar. 2, 2023

(54) SYSTEMS AND METHODS FOR A DEEP NEURAL NETWORK WITH COMBINED CROSS-ENTROPY AND FOCAL LOSS FOR COVID-19 DETECTION IN AUDIO SAMPLES

(71) Applicants: Sunil Rao, Tempe, AZ (US); Vivek Sivaraman Narayanaswamy, Tempe, AZ (US); Michael Esposito, Phoenix, AZ (US); Andreas Spanias, Tempe, AZ (US)

(72) Inventors: Sunil Rao, Tempe, AZ (US); Vivek Sivaraman Narayanaswamy, Tempe, AZ (US); Michael Esposito, Phoenix, AZ (US); Andreas Spanias, Tempe, AZ (US)

(73) Assignee: Arizona Board of Regents on Behalf of Arizona State University, Tempe, AZ (US)

(21) Appl. No.: 17/819,519

(22) Filed: Aug. 12, 2022

Related U.S. Application Data
(60) Provisional application No. 63/232,862, filed on Aug. 13, 2021.

(51) Int. Cl. A61B 5/08; G10L 25/18; G10L 25/30; G10L 25/66; A61B 5/00; G16H 50/70; G16H 50/20

(52) U.S. Cl. CPC A61B 5/0823; G10L 25/18; G10L 25/30; G10L 25/66; A61B 5/7267; A61B 5/7275; G16H 50/70; G16H 50/20

(57) ABSTRACT

Various embodiments of a system and associated method for detection of COVID-19 and other respiratory diseases through classification of audio samples are disclosed herein. The system utilizes features directly extracted from the coughing audio and develops automated diagnostic tools for COVID-19. In particular, the present application discusses a novel modification of a deep neural network architecture by using log-mel spectrograms of the audio excerpts and by optimizing a combination of binary cross-entropy and focal loss parameters. One embodiment of the system achieved an average validation AUC of 82.23% and a test AUC of 78.3% at a sensitivity of 80.49%.

### CROSS-REFERENCE TO RELATED APPLICATIONS

This is a U.S. Non-Provisional patent application that claims benefit to U.S. Provisional Patent Application Ser. No. 63/232,862 filed 13 Aug. 2021, which is herein incorporated by reference in its entirety.

### GOVERNMENT SUPPORT

This invention was made with government support under 1540040 awarded by the National Science Foundation. The government has certain rights in the invention.

### FIELD

The present disclosure generally relates to audio sample classification, and in particular, to a system and associated method for audio-based detection of respiratory diseases including COVID-19.

### BACKGROUND

With the outbreak of COVID-19 caused by the coronavirus SARS-CoV-2, the severity of the infection and the associated fatality rates around the world continue to increase at an overwhelming rate, motivating the need for rapid and reliable screening approaches. Although reverse transcriptase-polymerase chain reaction (RT-PCR) testing is commonly adopted, coughing sounds have been found to reveal useful signatures pertaining to COVID-19 which can be used to facilitate rapid, non-invasive and reliable screening strategies. Consequently, spectral and waveform signatures of the disease are being considered to design biomarkers for early diagnosis of the infection. In this context, there have been initiatives towards enabling open research on COVID-19 detection driven by cough sounds. For example, the curated Coswara dataset of cough samples collected from patients who tested COVID-19 positive as well as from patients who tested negative. Similarly, the curated dataset (“COUGHVID” dataset) consisting of crowd sourced cough samples collected from COVID positive and negative patients across a wide range of demographics. These efforts naturally pave the way to the development of automated diagnosis tools for detecting COVID-19 from cough samples.

It is with these observations in mind, among others, that various aspects of the present disclosure were conceived and developed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram showing a system for COVID-19 detection through classification of audio samples;

FIG. 2 is a graphical representation showing an audio sample split into segments and extraction of corresponding spectrograms of each segment of the audio sample using the system of FIG. 1;

FIG. 3 is a simplified diagram showing a deep neural network implementation for illustration purposes;

FIG. 4 is a graphical representation showing classification task results of the deep neural network of FIG. 3, note that pruning degraded the performance of the classification task;

FIG. 5 is a diagram showing a classification framework of the system of FIG. 1;

FIG. 6 is a diagram showing average validation AUC curve on a plurality of validation folds for evaluation of the system of FIGS. 1 and 5;

FIG. 7 is a diagram showing average validation confusion matrix for evaluation of the system of FIGS. 1 and 5;

FIGS. 8A and 8B are a series of process flow diagrams showing a method for implementation of the system of FIGS. 1 and 5; and

FIG. 9 is a simplified diagram showing an exemplary computing system for implementation of the system of FIGS. 1 and 5 and the method of FIGS. 8A and 8B.

Corresponding reference characters indicate corresponding elements among the view of the drawings. The headings used in the figures do not limit the scope of the claims.

### DETAILED DESCRIPTION

Given the complex nature of spectral or waveform signatures of cough sounds, there is a need for sophisticated representation-learning approaches that can effectively leverage the intricate dependencies and automatically identify key attributes in the data to make informed decisions. One such system is disclosed herein that aims to classify cough samples into two categories namely, healthy and COVID-19 positive. Specifically, in one embodiment, the system trains at least one classifier on time-frequency representations of an audio sample, and through carefully designed loss functions and model ensemble strategies combat the inherent challenge of class imbalance.

### I. Introduction

Initially, a deep neural network (DNN) model was trained on a set of training data using cross-entropy without up-sampling the set of training data. The present disclosure describes the initial DNN model having fully connected layers for cough detection, for comparison with the system described herein. With an intent to import such DNN models for COVID-19 cough detection on mobile and edge-devices, the initial DNN model was pruned based on the Lottery Ticket Hypothesis (LTH) optimization process. Even following pruning, it was observed that the model was initially biased towards a densely sampled healthy class and performed poorly on both validation and blind test sets.

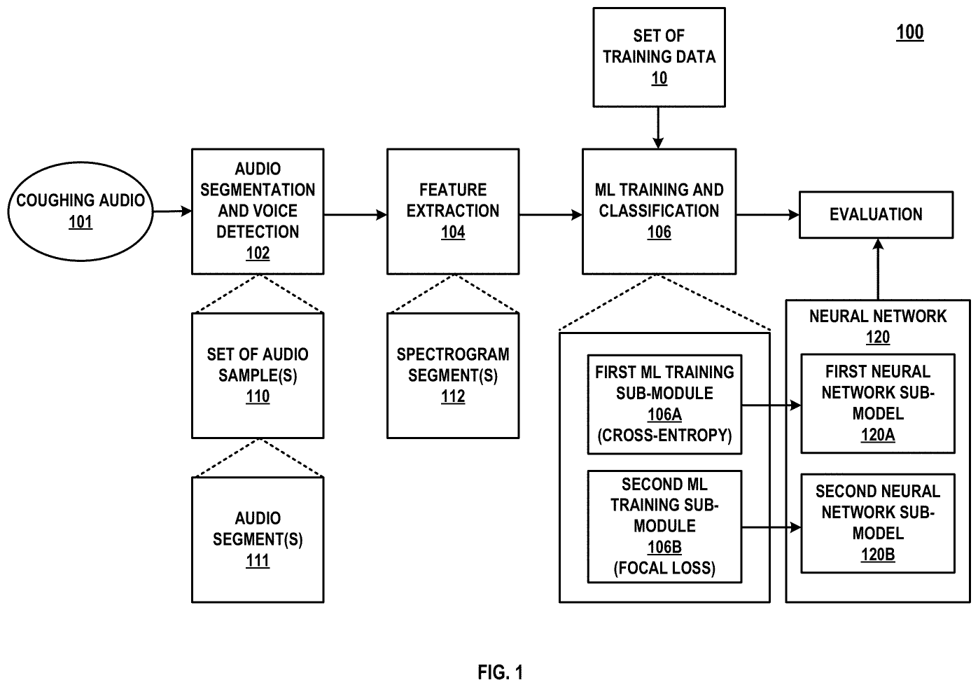

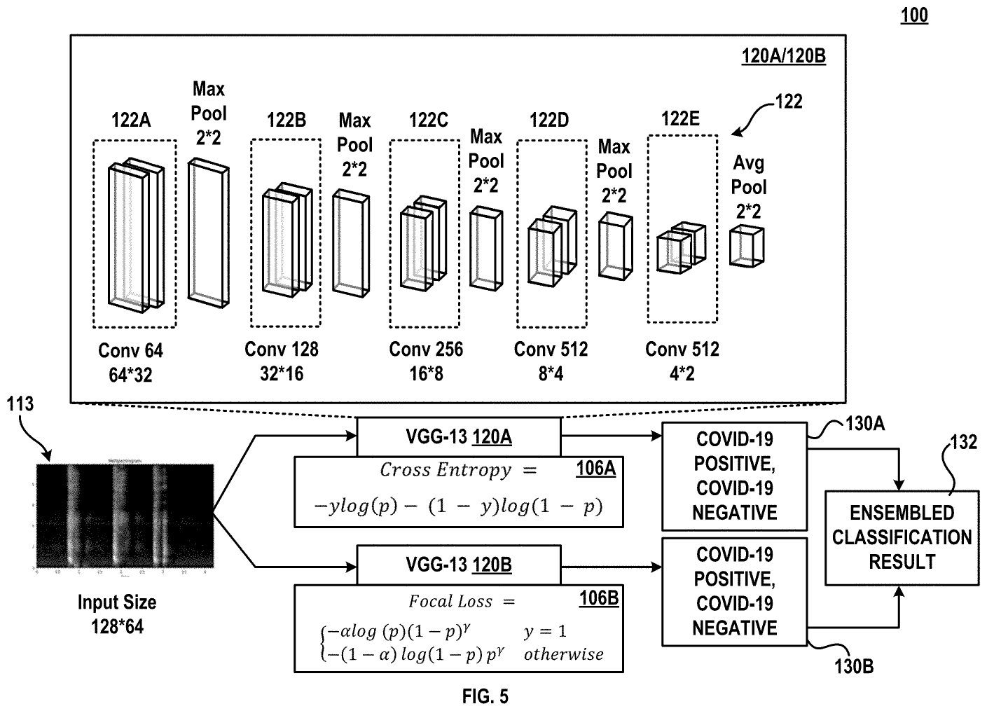

To improve these results, a system 100 described herein and shown in FIGS. 1 and 5 incorporates additional COVID-19 examples from an external dataset (COUGHVID dataset), upsamples a set of training data 10 and then trains a neural network to identify COVID cases from audio data by optimizing cross-entropy loss and focal loss parameters. The system 100 incorporates an ensemble of neural network models (VGG-13 convolutional neural networks), where a first neural network sub-model is trained using cross-entropy and upsampling and a second neural network sub-model is trained using focal loss; the system 100 combines classification results from the first neural network sub-model and the second neural network sub-model to yield an ensembled classification result.

Initially, the system 100 only incorporated cross-entropy during training. While the performance improved on the validation data compared to the initial DNN model, performance on the blind dataset was still low. To improve generalization of the neural network and to ensure that the model predictions are better calibrated, the system 100 incorporates focal loss during training. Focal loss tuning addresses the class imbalance problem by penalizing a class with a higher number of samples during training. During experimentation, different weighting strategies were explored for combining the models trained on cross-entropy and focal loss functions. However, it was noticed that assigning equal weights to each model during prediction on the validation and test dataset gave desirable results. Various features were also explored for audio. It was determined through experimentation that using log-mel spectrogram representations for the audio sample provided the best results.

### II. Pre-Processing and Feature Extraction

Training Dataset

The set of training data 10 includes a plurality of training audio samples, where one or more training audio samples include a class label. Each training audio sample of the set of training data 10 can be classified into a first class indicative of a presence of a disease (e.g., COVID-19 positive) or a second class indicative of no presence of the disease (e.g., COVID-19 negative). Note that while the present application is discussed in terms of classifying audio samples with respect to COVID-19 status, the system 100 can be extended to classify audio samples with respect to other diseases or infections such as pertussis (whooping cough), chronic pulmonary obstructive disease (COPD), influenza, and other pathologies.

The DiCOVA Challenge was designed to find scientific and engineering insights on COVID-19 by analyzing acoustic cues gathered from COVID-19 positive and non-COVID-19 individuals. The goal is to use cough sound recordings from COVID-19 and non-COVID-19 individuals for the task of COVID-19 detection; these cough sound recordings were used as a portion of the set of training data 10. The DiCOVA dataset includes a total of ˜1.36 hours of cough audio recordings from 75 COVID-19 positive subjects and 965 COVID-19 negative subjects. Out of these samples, there were a total of five splits for training and validation. The challenge also required the participants to evaluate the models on a blind test dataset.

Silence Removal and Downsampling

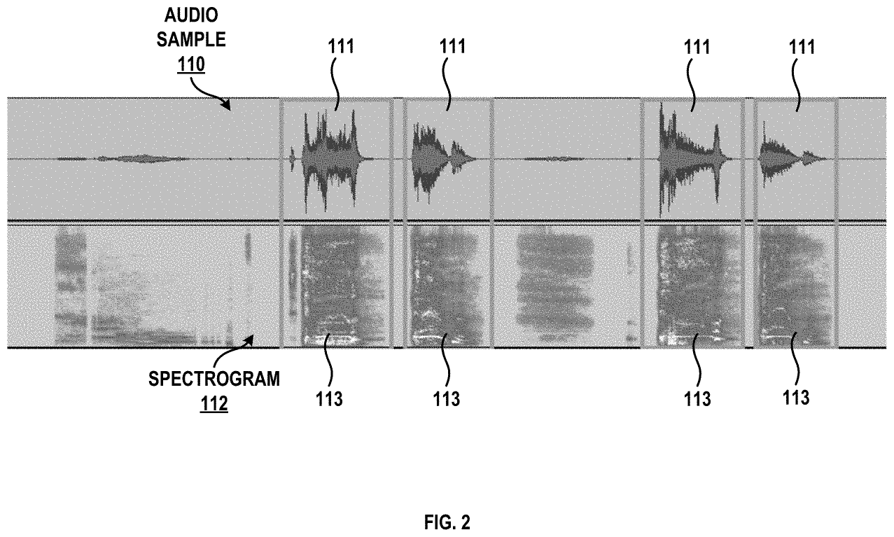

To classify an instance (e.g., an audio sample, where the audio sample can be classified into the first class indicative of the disease or the second class indicative of no presence of the disease), the system 100 first applies an audio segmentation process 102 on an audio file 101; for the specific application of identifying COVID-19 cases from audio data, the audio file 101 includes coughing audio. In particular, the audio segmentation process 102 involved applies a silence removal process on one or more given audio files 101 including one or more audio samples 110 shown in FIG. 2. If the duration of silence within the audio file 101 is found to be greater than 500 ms, the system 100 splits the audio file 101 at the corresponding instance. Similarly, if an amplitude of the audio file 101 is found to be less than −48 dB, the system 100 removes the corresponding section of the audio file 101. After the silence removal process, the system 100 performs a downsampling process of the audio file 101 to 32 kHz. A selected sampling rate was chosen to be 32 kHz to maintain uniform sampling rates across all datasets chosen to train the neural network 120 of the system 100 for classification of samples within the audio file 101; the pre-processing steps yield a set of audio samples 110.

Feature Extraction

Following pre-processing, the system 100 applies a feature extraction process 104 to the set of audio samples 110; in particular, the feature extraction process 104 transforms the set of audio samples 110 to a spectral log-mel scale, yielding a set of log-mel spectrogram data 112 having a plurality of spectrogram segments 113. Log-mel features are suitable for classification tasks using neural networks, as they benefit from additional information such as rich temporal and spectral structure of the original signal. The set of audio samples 110 includes a plurality of audio segments 111; in one embodiment, each respective audio segment 111 of the audio sample 110 is selected to have a duration of 1 s. If any audio segment 111 of the plurality of audio segments 111 is less than 1 s long, the log-mel spectrogram data 112 is zero-padded. In some embodiments, a window size of 512 and hop size of 256 is selected to correspond to the 1 s spectrogram. Therefore, each spectrogram segment 113 of the set of log-mel spectrogram data 112 has a size of 128×64. Table 1 shows the parameters used to obtain the set of log-mel spectrogram data 112. FIG. 2 shows an audio file being split into the plurality of audio segments 111 each having a duration of 1 second, and the extraction of the spectrogram segment 113 of the set of log-mel spectrogram data 112 for each respective audio segment 111 of the plurality of audio segments 111. The spectrogram segments 113 obtained each have a size of 128×64 and are passed as an input to the neural network 120.

TABLE 1
Parameters used to obtain the Log-Mel Spectrogram.

| Parameter | Configuration |
|---|---|
| Sample Rate | 32000 Hz |
| Window Size | 512 |
| Hop Size | 256 |
| Mel Bands | 64 |

Experimentation with Additional Features

Prior to selection of the log-mel spectrogram for implementation within the present system, the use of classical features was investigated, such as the Frame level mel-frequency cepstral coefficients (MFCC) with the delta and delta-delta co-efficients, Root-Mean-Square (RMS), Spectral Centroid (SC), Spectral Roll-off (SR), Spectral-Bandwidth (SB) and the Zero-Crossing Rate (ZCR) in classifying the cough sounds as either belonging to a COVID-19 positive or a healthy patient. In particular, these statistical features were concatenated and an XGBoost model was utilized to perform the classification. However, it was found that despite using sophisticated model selection strategies; namely, 5-fold cross validation and hyper-parameter tuning, the generalization even to the validation data was not sufficient. It was found that the model predicted almost all the COVID-19 positive samples as belonging to the healthy class. It was also observed that the other classification metrics (AUROC, Specificity) were found to be 55% and 40% respectively which naturally motivated the choice for a more powerful feature extractor. Similar performances were observed with the use of a feed-forward neural network trained with these features. In the following sections, the present disclosure describes the use of a fully connected neural network for COVID-19 cough detection where the neural network is trained using the log-mel spectrograms. Particularly, section III describes the initial DNN model that was investigated, section IV describes modification of the initial DNN model (through pruning based on Lottery Ticket Hypothesis), and the following section V describes the system 100 incorporating the ensemble of neural network models (VGG-13 convolutional neural networks), where the system 100 combines classification results from a first neural network sub-model (trained using cross-entropy) and a second neural network sub-model (trained using focal loss) to yield an ensembled classification result. The system 100 described in section V outperforms the initial DNN model discussed in sections III and IV; the discussion in sections III and IV is provided for illustration and comparison purposes.

Ill. Detectors Based on Fully Connected Neural Networks

In this disclosure, COVID-19 cough detection is considered as a supervised learning task. For a fully connected DNN, consider a dataset {X, y} where X ∈ R^{m×n} where m is the total number of samples and n is the input feature dimension; y ∈ {0,1} is the target label of whether a sample belongs to a COVID-19 negative (0) or a COVID-19 positive (1) class. In order to feed the cough audio spectrograms into a fully connected network, the spectrograms are first vectorized (flattened) and stacked to obtain a dataset X ∈ R^{6385×8192}. The input spectrograms are fed as inputs to a fully connected DNN, popularly referred to as the multi-layer perceptron (MLP). The MLP model can be a 5 layered neural network of hidden layer dimensions 50, 100, 500, 1000 respectively. Information flows through the neural network in two ways: (i) In forward propagation, the MLP model predicts the output for the given data and (ii) In backpropagation, the model adjusts its parameters considering the error in the prediction. The activation function used in each neuron allows the MLP to learn a complex function mapping. Input to the model is the feature vector x ∈ X, the output of the first and consecutive hidden layer is given by:

h₁=σ(W₁·x+b₁) (1)

hᵢ=σ(Wᵢ·x+bᵢ) (2)

where i is the layer index and σ is the activation function. The final output of the MLP is obtained as:

ŷ=φ_{softmax}(hₒᵤₜ) (3)

Weights of each neuron are trained/updated using a scaled gradient backpropagation algorithm. Each layer is assigned a tanh (hyperbolic tangent) activation function. From experimentation, it was found that the tanh activation function provided the best accuracy. The output layer uses the SoftMax activation function to categorize the type of cough in the given dataset. The given training and validation splits of the DiCOVA dataset to train and evaluate the model. Using a fully connected DNN, an average accuracy of ˜65% was observed on the given validation splits. Consequently, the act of pruning the fully connected DNN was explored to develop sparse or compressed neural networks to understand whether similar performance gains can be expected. The use of sparse neural networks can be potentially used in development of COVID-19 detection software on hardware and cloud applications.

### IV. Pruned Neural Networks Using Lottery Ticket Hypothesis

Pruned neural networks (NNs) on embedded hardware significantly provides computational efficiencies and reduce memory requirements with only with slight reduction in accuracy in comparison with the unpruned networks. One suitable strategy to prune a NN is based upon the Lottery Ticket Hypothesis, which is based upon the idea that a randomly initialized, dense neural network contains a subnetwork initialized such that, when trained in isolation, it can match the test accuracy of the original network after training for at most the same number of iterations. Consider a fully connected NN with N neurons in each layer initialized by weight matrices W⁰={Wᵢ⁰}ᵢ₌₁^{L}. After training this network for t epochs, the resulting weights of the network are W^{t}. Next, compute a mask M by pruning p % of the of weights closer to zero by taking the absolute value. Reinitialize the network with W⁰ masked by M. The network training and network pruning process is iterated until 2.5× compression is achieved, after which the networks performance degrades due to underfitting of the data.

Randomly initialize a neural network f(x; m ⊙ θ) where θ=θ₀ and m=1^{|θ|} is a mask.

Train the network for j iterations, reaching parameters m ⊙ θᵢ.

Prune s % of the parameters, creating an updated m′ where P_{m′}=(Pₘ−s) %.

Reset the weights of the remaining portion of the network to their values in θ₀. That is, let θ=θ₀.

Let m=m′ and repeat steps 2 to 4 until a sufficiently pruned network has been obtained.

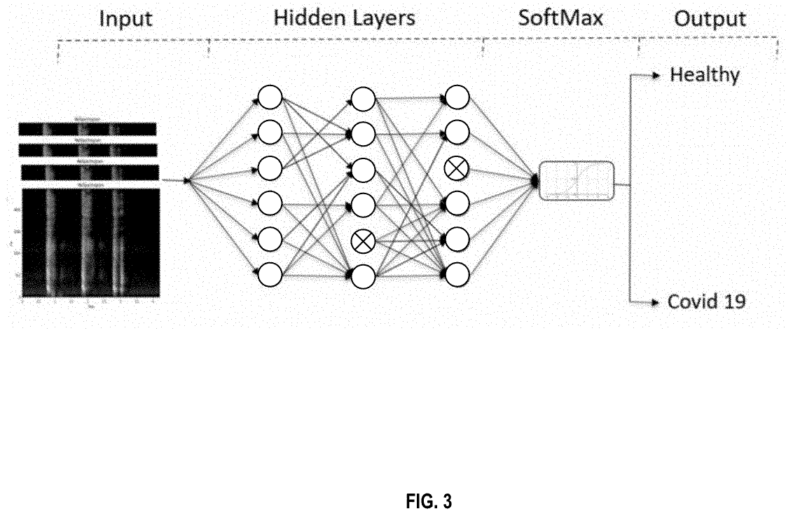

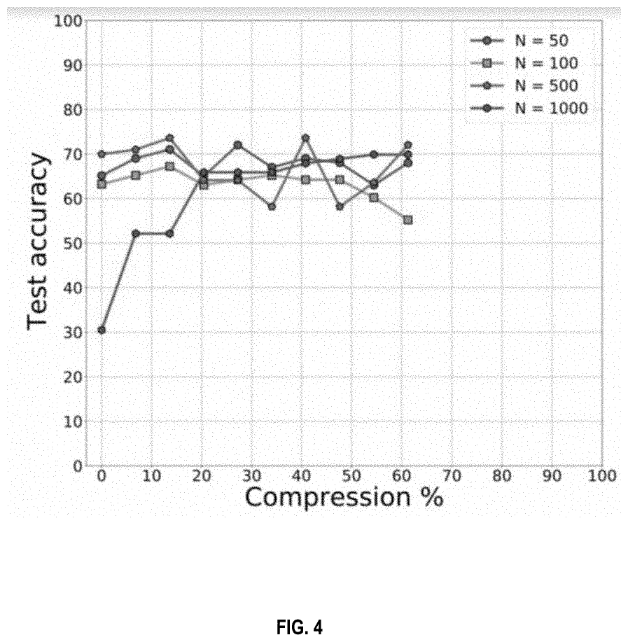

For the network pruning experiments, NNs with four hidden layers each with N={50, 100, 500, 1000} neurons (FIG. 3) were considered. All NNs were trained for 150 epochs and at every pruning iteration 10% of the remaining weights were pruned. It was found that pruning a network for this setup degrades the performance of the classification task as shown in FIG. 4. While utilizing fully connected neural networks, the inputs are essentially vectors of the spectrogram samples. The architecture of these NNs is such that they do not consider local spatio-temporal and periodic patterns highly prevalent in audio spectrogram data. Therefore, in order to exploit these patterns, the use of deep convolutional neural network architectures is explored which by design can identify markers in audio critical to distinguish between COVID-19 negative and COVID-19 positive cough sounds.

### V. Ensembled CNN Architectures

The system 100 includes an ML training and classification module 106 for training the neural network 120. Referring to ML training and classification module 106 of FIGS. 1 and 5, a convolutional neural network (CNN) architecture (e.g., a VGG-13 neural network) was used to implement as the neural network 120; the ML training and classification module 106 to trains and evaluates performance of the neural network 120 over different training and validation splits. In particular, a VGG-13 neural network used as the basis for neural network 120 includes multiple convolutional blocks followed by a fully connected network to make the final predictions. The inputs to the neural network 120 include are the spectrogram segments 113 of the set of log-mel spectrogram data 112 generated from each audio file 101 with size 128×64. Each convolutional block includes two convolutional layers followed by a max pooling layer that halves each of the spatial dimensions. Every convolution layer is followed by rectified linear unit (ReLU) activation function and batch normalization which is a form of regularization to tackle internal covariate shifts. After the convolutional blocks, resulting feature maps are average pooled and fed as inputs to a feed-forward neural network. Finally, a SoftMax layer is applied to generate the predictions. The size of the spectrogram that is used as input to the VGG network is 128×64. Spectrograms were generated from each audio excerpt after suitable pre-processing.

More details related to the neural network 120 and the input features are provided in the subsequent sections. It should be noted that the neural network 120 can be modified with adjusted hyperparameters for optimized performance of the ML training and classification module 106. FIG. 5 shows an overview of the ML training and classification module 106 (shown in FIG. 5 as a first ML training sub-module 106A and a second ML training sub-module 106B) and the neural network 120 (shown in FIG. 5 as a first neural network sub-model 120A and a second neural network sub-model 120B).

The neural network 120 (specifically, the first neural network sub-model 120A and the second neural network sub-model 120B) includes a plurality of convolutional blocks 122A-122E and takes a spectrogram segment 113 of the set of log-mel spectrogram data 112 as input. Each respective convolutional block 122A-122E of the plurality of convolutional blocks 122 includes two convolutional layers followed by a max pooling layer that halves each spatial dimension (where dimensions of the first convolutional block 122A are 64*32, the second convolutional block 122B are 32*16, the third convolutional block 122C are 16*8, the fourth convolutional block 122D are 8*4 and the fifth convolutional block 122E are 4*2). After each respective convolution, which uses the ReLU activation function, batch normalization is applied as a form of regularization. After the convolutional blocks 122A-122E, each respective channel is averaged to a scalar value. Finally, a softmax layer is applied to generate the prediction in the form of first classification value 130A and a second classification value 130B that are each indicative of a respective first and second classification prediction yielding an ensembled classification result 132 for the audio sample 110, where the ensembled classification result 132 is indicative of a first class indicative of a presence of a disease or a second class indicative of no presence of the disease. In some embodiments, the disease is COVID-19; as such, the set of training data 10 includes training audio samples that are classified as COVID-19 positive or COVID-19 negative and the ensembled classification result 132 for the audio sample 110 can indicate the first class (e.g., COVID-19 positive) or the second class (e.g., COVID-19 negative). Note that the system 100 can be extended to classify the audio sample 110 according to a selected pathology; the set of training data 10 used to train the first neural network sub-model 120A and the second neural network sub-model 120B would need to include examples with respect to the selected pathology.

Due to the imbalance in the sampling density across the healthy and COVID-19 positive classes within the set of training data 10, the neural network 120 includes the first neural network sub-model 120A trained using cross-entropy by the first ML training sub-module 106A that yields the first classification value 130A and the second neural network sub-model 120B trained using focal loss by the second ML training sub-module 106B that yields the second classification value 130B. The ML training and classification module 106 of the system 100 augments Di-COVA dataset with the COUGHVID dataset to form the set of training data 10 to train the neural network 120, and upsamples the set of training data 10 to maintain data balance. The first ML training sub-module 106A of the ML training and classification module 106 trains the first neural network sub-model 120A of the neural network 120 using a cross-entropy loss on the set of training data 10, while the second ML training sub-module 106B of the ML training and classification module 106 trains the second neural network sub-model 120B of the neural network 120 on the set of training data 10 using a focal loss to address data imbalance.

Data Augmentation

Since the DiCOVA 2021 dataset has only 50 COVID-19 positive samples per validation fold, the ML training and classification module 106 augments the dataset with the COUGHVID dataset to form the set of training data 10 for training the first neural network sub-model 120A and the second neural network sub-model 120B of the neural network 120. In one embodiment, 400 COVID-19 positive samples were used from the COUGHVID dataset for network training. To avoid overfitting during training, the ML training and classification module 106 implements an additional data augmentation step in the form of an audio mixup method. The audio mixup method implemented by the ML training and classification module 106 randomly mixes a pair of inputs and their corresponding outputs. Consider a pair of inputs x₁ and x₂ with y₁ and y₂ being their corresponding labels. The audio mixup method uses a parameter λ ∈ (0, 1) to create a convex combination as shown below:

x=λx₁+(1−λ)x₂ (4)

y=λy₁+(1−λ)y₂ (5)

The output of equation (4) and equation (5) were used to train the first neural network sub-model 120A and the second neural network sub-model 120B of the neural network 120 rather than the original inputs. The parameter λ was chosen to be a random variable from the uniform distribution U˜(0, 1), therefore a different value of λ was used for each pair of inputs.

Loss Functions

The first neural network sub-model 120A and the second neural network sub-model 120B were based on based on two different (dataset, loss function) choices. The first neural network sub-model 120A is trained on cross-entropy loss and the second neural network sub-model 120B is trained on focal Loss. The outputs of the first neural network sub-model 120A and the second neural network sub-model 120B were combined to generate the probabilities on the validation and test datasets.

Cross-entropy Loss: To address data imbalance, the ML training and classification module 106 augments the COUGHVID dataset and upsamples the set of training data 10 for each training fold. A number of samples is balanced in both classes in the set of training data 10 through the upsampling step. The upsampled training data is used to train the first neural network sub-model 120A using the cross-entropy loss. The binary cross-entropy loss is given as:

CE=−y log(p)−(1−y)log(1−p) (6)

where y[0, 1] corresponds to the label of the two classes and p is the probability of the outputs.

Focal Loss: Focal loss is used to address the class imbalance problem. For a second neural network sub-model 120B, the second neural network sub-model 120B is modified by incorporation of two modulation hyperparameters α and γ−y to the cross-entropy loss shown in equation 6 to make the second neural network sub-model 120B more efficient and enable the second neural network sub-model 120B to handle classes with fewer numbers of samples as shown in equation 7. All 400 COVID-19 positive samples from COUGHVID are included to each training fold containing 50 DiCOVA COVID-19 positive samples. The focal loss is then used with modulation parameters α=0.25 and γ=2 to train the second neural network sub-model 120B.

$$FL = \{ \begin{matrix}- α log ( p ) {( 1 - p )}^{γ} & y = 1 \\ - ( 1 - α ) log ( 1 - p ) {p}^{γ} & otherwise\end{matrix} \qquad ( 7 )$$

Ensemble: The first neural network sub-model 120A and the second neural network sub-model 120B were respectively trained with the cross-entropy and focal loss functions described above. The individual models predict on the five validation folds and on the test dataset to each obtain a first value indicative of a first classification value 130A (from the first neural network sub-model 120A) and a second classification value 130B (from the second neural network sub-model 120B) for each validation/test sample. The ensembled classification result 132 is obtained for all validation/test samples by averaging the first classification value 130A from the first neural network sub-model 120A and the second classification value 130B from the second neural network sub-model 120B. As discussed above, in some embodiments, it was found that equal weighting between the first classification value 130A and the second classification value 130B yielded better classification results.

### Results

Benchmarking Different Design Strategies

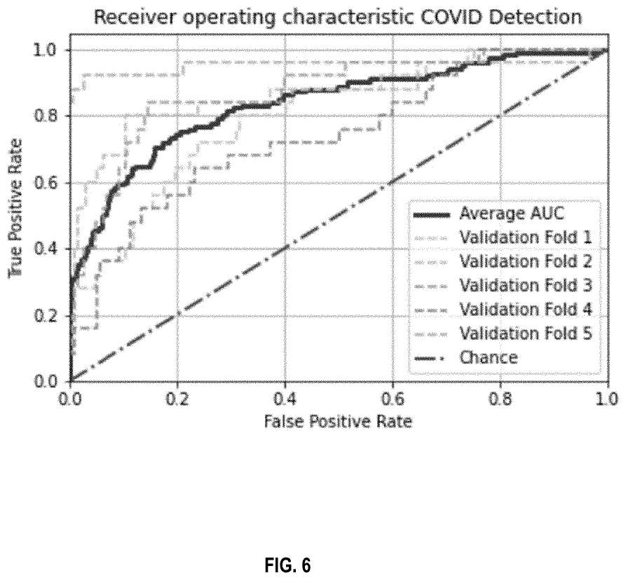

For the fully connected neural networks, an average validation accuracy of 65% was obtained on the 5 folds provided. In case of the pruned neural networks for cough detection, a similar classification accuracy of 64.3% is observed. It was found that smaller networks achieve greater compression of about 60% for a drop in accuracy by 7%. More sophisticated convolutional neural network architectures such as the VGG-13 were then explored, which were ultimately used in the system 100. This section first discuss the results obtained from VGG-13 models before and after ensembling. The first neural network sub-model 120A was trained on DiCOVA data augmented with COUGHVID data for each of the five folds with cross-entropy loss. An average AUC of 84.02% were obtained on the five folds. The test AUC was found to be 73.48%. For the second neural network sub-model 120B the training was repeated without upsampling; however, the second neural network sub-model 120B was trained using focal loss as the loss function. An average validation AUC of 72.01% was reported on the five folds. The test AUC was found to be 73.44%. These results are summarized in Table 2. Since the first neural network sub-model 120A and the second neural network sub-model 120B individually reported low AUC on the test dataset, the probability scores on the best of the first neural network sub-model 120A and the second neural network sub-model 120B were obtained. For example, to obtain an improved AUC score, the best first neural network sub-model 120A and the best second neural network sub-model 120B were selected and ensembled for each loss function and from each fold for predicting the corresponding validation fold to obtain the ensembled AUC. The test probability scores were also based on the ensembled models. The average validation AUC was found to be 82.23% (as observed from FIG. 6) on the validation folds, while the AUC on the blind test dataset was found to be 78.3%.

TABLE 2
AUC for each individual fold of the validation
dataset with combined cross-entropy loss and focal
loss with highest specificity for each fold.

| | AUC under | AUC under |
|---|---|---|
| Validation | Cross-entropy | Focal Loss |
| Fold | (%) | (%) |
| 1 | 80.71 | 70.27 |
| 2 | 89.88 | 68.76 |
| 3 | 87.12 | 77.78 |
| 4 | 73.59 | 70.52 |
| 5 | 94.92 | 72.93 |

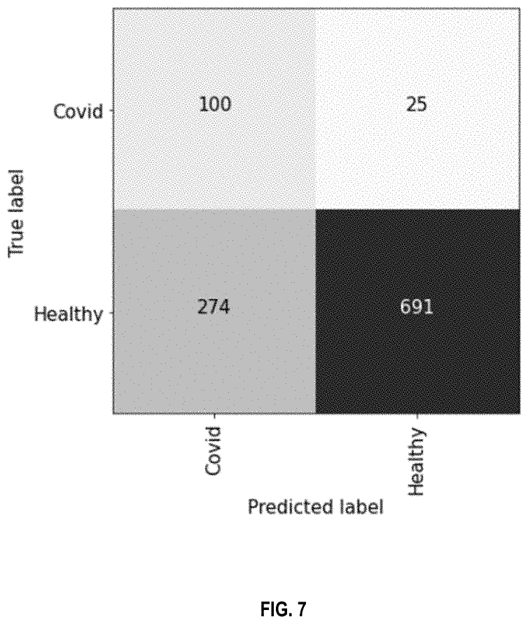

FIG. 7 depicts the confusion matrix obtained at a sensitivity of 80%, which implies that 80% of the positive class was correctly detected. The validation scores were obtained from each fold and the overall performance was reported herein. One can observe that at such a high sensitivity, the number of false negatives is significantly less than the total number of true negatives, which is indicative of the detection performance.

Comparison with Methods from the Interspeech DiCOVA 2021 Challenge

The system 100 implementing the neural network 120 (using the modified VGG-13 classifier) was entered into the “Diagnosing COVID-19 Using Acoustics” (DiCOVA) 2021 challenge, a special session of Interspeech 2021. Participants were tasked with a COVID-19 audio classification task involving a blind test set of 233 audio samples. The system 100 placed eleventh out of 85 teams in the Track-1 challenge. In this section, methodologies and results for a number of the participants are described for illustration and comparison with the present system 100. All AUROC values given are for performance on the 5-fold cross validation. One team developed a method using Teager energy operator cepstral coefficients (TECCs) and a light gradient boosting machine (LightGBM) to achieve an AUROC of 69.80%. Another team developed a method using support vector machines (SVMs) and long short-term memory (LSTM) networks in order to achieve and AUROC of 94.31%. Another team used Random Forests, and SVMs in conjunction with the ComParE 2016 feature set. An AUROC of 73.29% was attained using this method. Another study demonstrated the use of logistic regression, random forests, and multilayer perceptrons to obtain an AUC value of 73.41%. In another study, autoregressive predictive coding (APC) was used in addition to an LSTM to achieve an AUC value of 76.45%. Another system only achieved an AUC of 47.28% but used a unique encoder-decoder methodology for COVID-19 cough classification. In another system, a weighted SVM was used with audio features such as super-vectors, formants, harmonics and MFCCs to attain an AUROC of 71.7%. In another system, a Resnet18 model was implemented for classification and evaluate performance of their algorithm between samples from male and female participants. This algorithm achieved an AUC of 61.90%. Another study reported an AUROC of about 73.4% using handcrafted features and an SVM classifier. Another team implemented an SVM classifier with high-dimensional acoustic features, as well as a CNN with log-Mel spectrograms. These models were fused in order to achieve an AUROC of 81%. The present system 100 can perform COVID-19 detection with an average AUROC of 82.23%, identifying COVID-19 samples with an accuracy of 80% and a false positive rate of 17%.

Conclusion

To summarize, the system 100 includes a modified VGG-13 neural network 120 with combined cross-entropy and focal loss, operating on the Log-mel spectrogram to learn to discriminate between the spectral signatures of coughs from a finite set of COVID-19 positive and healthy patients. The overall generalization performance was improved by: (i) performing data-augmentation using the COVID-19 positive cough sounds from the crowd sourced COUGHVID dataset; and (ii) using ensembles of two VGG-13 models, where the former was trained using the cross-entropy loss and the latter was trained using the focal loss. With these key modifications to the predictive modeling pipeline, the present approach achieved high validation AUROCs (82.23%) and blind test AUROCs (78.3%), thereby encouraging the potential use of such models to support rapid diagnosis in clinical settings.

### Method

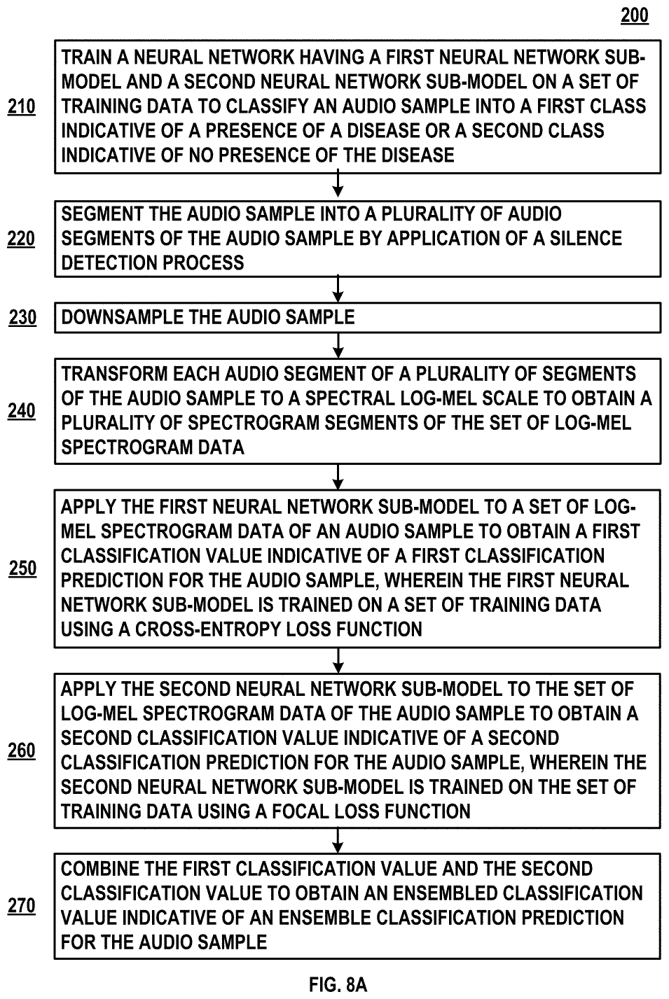

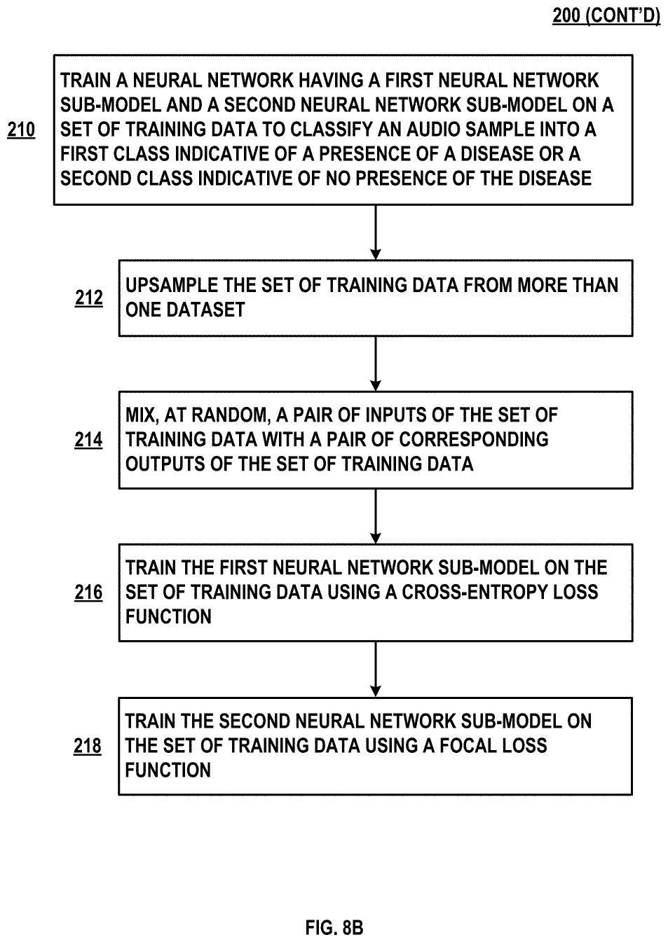

FIGS. 8A and 8B are a pair of process flow diagrams showing a method 200 for audio classification according to the system 100 of FIGS. 1 and 5.

Method 200 starts at block 210 (FIGS. 8A and 8B) that includes training a neural network having a first neural network sub-model and a second neural network sub-model on a set of training data to classify an audio sample into a first class indicative of a presence of a disease or a second class indicative of no presence of the disease. Block 210 can include various sub-blocks shown in FIG. 8B, including block 212 which includes upsampling the set of training data from more than one dataset, block 214 which includes mixing, at random, a pair of inputs of the set of training data with a pair of corresponding outputs of the set of training data, block 216 which includes training the first neural network sub-model on the set of training data using a cross-entropy loss function, and block 218 which includes training the second neural network sub-model on the set of training data using a focal loss function. Note that steps outlined in block 216 and block 218 can be performed simultaneously.

Referring back to FIG. 8A, block 210 is followed by block 220, which includes segmenting the audio sample into a plurality of audio segments of the audio sample by application of a silence detection process. Block 230 includes downsampling, at the processor, the audio sample. Block 240 includes transforming each audio segment of a plurality of segments of the audio sample to a spectral log-mel scale to obtain a plurality of spectrogram segments of a set of log-mel spectrogram data. Blocks 220-240 can be collectively described as pre-processing steps that result in the set of log-mel spectrogram data of the audio sample, which are then provided as input to the first neural network sub-model and the second neural network sub-model.

Block 250 includes applying the first neural network sub-model to the set of log-mel spectrogram data of the audio sample to obtain a first classification value indicative of a first classification prediction for the audio sample. Block 260 similarly includes applying the second neural network sub-model to the set of log-mel spectrogram data of the audio sample to obtain a second classification value indicative of a second classification prediction for the audio sample. Note that steps outlined in block 250 and block 260 can be performed simultaneously. Block 270 includes combining the first classification value and the second classification value to obtain an ensembled classification value indicative of an ensemble classification prediction for the audio sample.

### Computer-Implemented System

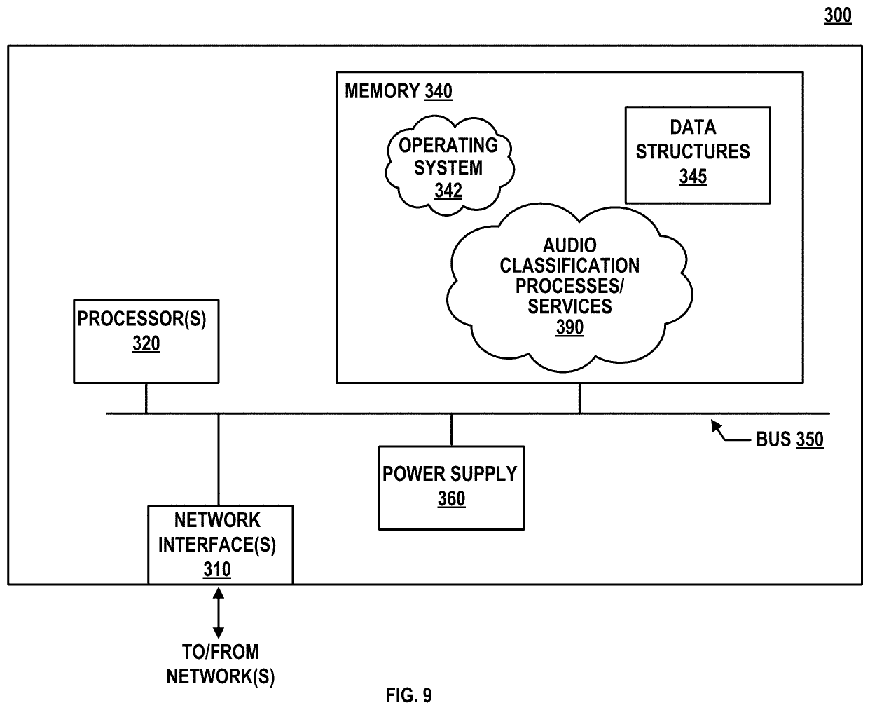

FIG. 9 is a schematic block diagram of an example device 300 that may be used with one or more embodiments described herein, e.g., as a component of system 100 and/or as a computing device for implementation of aspects of system 100 shown in FIGS. 1 and 5 and method 200 shown in FIGS. 8A and 8B.

Device 300 comprises one or more network interfaces 310 (e.g., wired, wireless, PLC, etc.), at least one processor 320, and a memory 340 interconnected by a system bus 350, as well as a power supply 360 (e.g., battery, plug-in, etc.).

Network interface(s) 310 include the mechanical, electrical, and signaling circuitry for communicating data over the communication links coupled to a communication network. Network interfaces 310 are configured to transmit and/or receive data using a variety of different communication protocols. As illustrated, the box representing network interfaces 310 is shown for simplicity, and it is appreciated that such interfaces may represent different types of network connections such as wireless and wired (physical) connections. Network interfaces 310 are shown separately from power supply 360, however it is appreciated that the interfaces that support PLC protocols may communicate through power supply 360 and/or may be an integral component coupled to power supply 360.

Memory 340 includes a plurality of storage locations that are addressable by processor 320 and network interfaces 310 for storing software programs and data structures associated with the embodiments described herein. In some embodiments, device 300 may have limited memory or no memory (e.g., no memory for storage other than for programs/processes operating on the device and associated caches). Memory 340 can include instructions executable by the processor 320 that, when executed by the processor 320, cause the processor 320 to implement aspects of the system 100 and the method 200 outlined herein.

Processor 320 comprises hardware elements or logic adapted to execute the software programs (e.g., instructions) and manipulate data structures 345. An operating system 342, portions of which are typically resident in memory 340 and executed by the processor, functionally organizes device 300 by, inter alia, invoking operations in support of software processes and/or services executing on the device. These software processes and/or services may include audio classification processes/services 390 described herein that implement aspects of method 200. Note that while audio classification processes/services 390 is illustrated in centralized memory 340, alternative embodiments provide for the process to be operated within the network interfaces 310, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the audio classification processes/services 390 is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

1. A system, comprising:
a processor in communication with a memory, the memory including instructions, which, when executed, cause the processor to:
apply a first neural network sub-model to a set of log-mel spectrogram data of an audio sample to obtain a first classification value indicative of a first classification prediction for the audio sample, wherein the first neural network sub-model is trained on a set of training data using a cross-entropy loss function;
apply a second neural network sub-model to the set of log-mel spectrogram data of the audio sample to obtain a second classification value indicative of a second classification prediction for the audio sample, wherein the second neural network sub-model is trained on the set of training data using a focal loss function; and
combine the first classification value and the second classification value to obtain an ensembled classification result indicative of an ensembled classification prediction for the audio sample, the ensembled classification prediction being indicative of a first class indicative of a presence of a disease or a second class indicative of no presence of the disease.

2. The system of claim 1, wherein the cross-entropy loss function is given by:
CE=−y log(p)−(1−y)log(1−p).
wherein y ∈ {0, 1} corresponds to a respective classification label and p corresponds to the first classification value.

3. The system of claim 1, wherein the focal loss function is given by:
$$FL = \{ \begin{matrix}- α log ( p ) {( 1 - p )}^{γ} & y = 1 \\ - ( 1 - α ) log ( 1 - p ) {p}^{γ} & otherwise\end{matrix}$$
wherein α and γ are each a modulation hyperparameter.

4. The system of claim 1, wherein the set of log-mel spectrogram data includes a plurality of spectrogram segments of the set of log-mel spectrogram data, each spectrogram segment of the plurality of spectrogram segments corresponding to a respective audio segment of a plurality of audio segments of the audio sample.

5. The system of claim 1, wherein the memory further includes instructions, which, when executed, further cause the processor to:
transform each audio segment of a plurality of segments of the audio sample to a spectral log-mel scale to obtain a plurality of spectrogram segments of the set of log-mel spectrogram data.

6. The system of claim 1, wherein the memory further includes instructions, which, when executed, further cause the processor to:
segment the audio sample into a plurality of audio segments of the audio sample by application of a silence detection process; and
downsample the audio sample.

7. The system of claim 1, wherein the memory further includes instructions, which, when executed, further cause the processor to:
combine the first classification value and the second classification value by averaging the first classification value and the second classification value into the ensembled classification result.

8. The system of claim 1, wherein the set of training data includes a plurality of audio samples, wherein each audio sample of the plurality of audio samples includes a classification label.

9. The system of claim 1, wherein the memory further includes instructions, which, when executed, further cause the processor to:
augment the set of training data by mixing, at random, a pair of inputs of the set of training data with a pair of corresponding outputs of the set of training data.

10. The system of claim 1, wherein the memory further includes instructions, which, when executed, further cause the processor to:
upsample the set of training data from more than one dataset.

11. The system of claim 1, wherein the memory further includes instructions, which, when executed, further cause the processor to:
train the first neural network sub-model and the second neural network sub-model on the set of training data to classify the audio sample into a first class indicative of a presence of a disease or a second class indicative of no presence of the disease;
wherein the set of training data includes a plurality of training samples, each training sample of the plurality of training samples being classified into the first class or the second class.

12. A method, comprising:
applying, at a processor in communication with a memory, a first neural network sub-model to a set of log-mel spectrogram data of an audio sample to obtain a first classification value indicative of a first classification prediction for the audio sample, wherein the first neural network sub-model is trained on a set of training data using a cross-entropy loss function;
applying, at the processor, a second neural network sub-model to the set of log-mel spectrogram data of the audio sample to obtain a second classification value indicative of a second classification prediction for the audio sample, wherein the second neural network sub-model is trained on the set of training data using a focal loss function; and
combining, at the processor, the first classification value and the second classification value to obtain an ensembled classification result indicative of an ensembled classification prediction for the audio sample, the ensembled classification prediction being indicative of a first class indicative of a presence of a disease or a second class indicative of no presence of the disease.

13. The method of claim 12, wherein the cross-entropy loss function is given by:
CE=−y log(p)−(1−y)log(1−p)
wherein y ∈ {0, 1} corresponds to a respective classification label and p corresponds to the first classification value.

14. The method of claim 12, wherein the focal loss function is given by:
$$FL = \{ \begin{matrix}- α log ( p ) {( 1 - p )}^{γ} & y = 1 \\ - ( 1 - α ) log ( 1 - p ) {p}^{γ} & otherwise\end{matrix}$$
wherein α and γ are each a modulation hyperparameter.

15. The method of claim 12, wherein the set of log-mel spectrogram data includes a plurality of spectrogram segments, each respective spectrogram segment corresponding to a respective plurality of audio segments of the audio sample.

16. The method of claim 12, further comprising:
transforming each audio segment of a plurality of segments of the audio sample to a spectral log-mel scale to obtain a plurality of spectrogram segments of the set of log-mel spectrogram data.

17. The method of claim 12, further comprising:
segmenting, at the processor, the audio sample into a plurality of audio segments of the audio sample by application of a silence detection process; and
downsampling, at the processor, the audio sample.

18. The method of claim 12, further comprising:
combining the first classification value and the second classification value by averaging the first classification value and the second classification value into the ensembled classification result.

19. The method of claim 12, wherein the set of training data includes a plurality of audio samples, wherein each audio sample of the plurality of audio samples includes a classification label.

20. The method of claim 12, further comprising:
mixing, at the processor and at random, a pair of inputs of the set of training data with a pair of corresponding outputs of the set of training data.

21. The method of claim 12, further comprising:
upsampling the set of training data from more than one dataset.

22. The method of claim 12, further comprising:
training the first neural network sub-model and the second neural network sub-model on the set of training data to classify the audio sample into a first class indicative of a presence of a disease or a second class indicative of no presence of the disease;
wherein the set of training data includes a plurality of training samples, each training sample of the plurality of training samples being classified into the first class or the second class.
